(19) United States
(12) Patent Application Publication
(10) Pub. No.: US 2004/0087564 A1
(43) Pub. Date: May 6, 2004

(54) Delivery composition and method

(76) Inventors: D. Craig Wright, Pacific Grove, CA (US); John E. Mauk, Arlington, VA (US)

Correspondence Address: FISH & RICHARDSON P.C., 1425 K STREET, N.W., 11TH FLOOR, WASHINGTON, DC, 20005-3500, US

(21) Appl. No.: 10/284,465

(22) Filed: Oct. 31, 2002

(51) Int. Cl. A61K 31/573

(52) U.S. Cl. 514/179

(57) ABSTRACT

A composition which includes a membrane modulators is disclosed. The composition can be used in a wide range of therapies for delivering a membrane modulator which play an active function in regulating, controlling or causing a desired therapeutic effect to a target cell.

### TECHNICAL FIELD

The invention relates to membrane modulator composition and method of transmitting the membrane modulator into a cell.

### BACKGROUND

The transmission of a biologically active material to cell is an essential step in a wide range of therapies. Therapies which supply the cell with biologically active material include, for example, a drug, a sterol, an antibiotic and other biologically active therapeutic materials. While the introduction of a biologically active material can be desirable, there can be obstacles to overcome to successfully accomplish the delivery to the cell site. Transmission of a biologically active material to a cell can involve transferring the material via a delivery system from an extracellular site to an intracellular site while maintaining the activity of the material and avoiding damage to the target cell. A delivery system which allows transmission of a biologically active material to a cell, protects the biologically active material from deactivation, and allows for targeting of the material to a specific cell, can be desirable.

### SUMMARY

In one aspect, a composition includes an emulsion. The emulsion includes a hydrophobic phase and a hydrophilic phase. The hydrophobic phase includes a membrane modulator and a wall forming material.

In another aspect, a composition including a non-Newtonian fluid. The non-Newtonian fluid includes a hydrophobic phase in contact with a hydrophilic phase.

In another aspect, a method of making a composition includes liquefying a hydrophobic phase and mixing the hydrophobic phase with a hydrophilic phase to form a non-Newtonian fluid including a membrane modulator. Liquefying can include melting. Mixing can be accomplished using a high shear mixer. The hydrophobic phase can be liquefied to form a slurry. The hydrophilic phase can be added to the hydrophobic phase while mixing, or the hydrophobic phase can be added to the hydrophilic phase while mixing.

In another aspect, a method of delivering a biologically active molecule includes providing a composition and contacting the composition with a human body. The composition includes a hydrophobic phase and a hydrophilic phase. The hydrophobic phase includes a membrane modulator compound and a wall forming material. Contacting can include transdermal delivery, intramuscular delivery, subcutaneous delivery, mucosal delivery, oral delivery, intravenous delivery, time release delivery, or delivery through a needle having a 25 gauge or thinner bore needle.

The hydrophobic phase can include a biologically active compound, and a wall forming material. The biologically active compound can be a sterol, an animal sterol, or a plant sterol. In certain embodiments, the hydrophobic phase can include an amphiphile, a biologically active compound, and a wall forming material. The amphiphile can have a positive charge or a negative charge. The amphiphile can be a surfactant. The range of ratios of hydrophobic phase to hydrophilic phase can be from about 1:11.5 to about 11.5:1. In some embodiments, the hydrophobic phase can include a surfactant, a membrane modulator, and a charge source. The charge source can have a positive charge or a negative charge.

The surfactant can be polyoxyethylene sorbitan ester, polyoxyethylene fatty acid ether or sorbitan monoester.

The wall forming material can be polyoxyethylene sorbitan ester, polyoxyethylene fatty acid ether or sorbitan monoester.

In certain embodiments, the hydrophobic phase includes polyoxyethylene(20) sorbitan monooleate, stearyl alcohol, and testosterone.

The hydrophilic phase can include water, an alcohol and water mixture, or phosphate buffered saline.

The membrane modulator can be an antibiotic, an amino acid, a peptide, a sterol, an animal sterol, a plant sterol, a modified sterol, a sterol, a ring structure, a multi-ring structure, a drug, testosterone, progesterone, estrogen, cholesterol, dehydroepiandrosterone, fludrocortisone, cortisol, creatine, miconazole nitrate, aspirin, 2-metronidazole, or 5-metronidazole, ciprofloxacin, cefuroxime, cefatrizine, cefpodoxime, clarithromycin, loracarbef, azithromycin, cefixime, cefadroxil, amoxycillin, acyclovir, cyclosporin, cephalosporin, baclofen, beta-sitostanol (24-ethyl-5-cholestene-3-beta-ol), beta-sitostanol (24-ethyl-5-alpha-cholestane-3-beta-ol), campesterol, brassicasterol, or stigmasterol. The membrane modulator can be a compound of the formula:

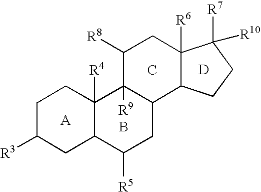

R³ can be hydroxy, acetoxy, propionoxy, methoxy, acyloxy, lower alkyloxy, benzoyloxy, or carbonyl. Each of R⁴ and R⁵, independently, can be hydrogen, methyl, lower alkyl, halo, alkoxy, or acyloxy. R⁶ can be methyl, lower alkyl, hydroxy, halo, alkoxy, or acyloxy. R⁷ can be hydrogen, carbonyl, halo, hydroxy, alkoxy, acyloxy, oxo, —C(O)CH₃, —C(O)CH₂OH or —CH(CH₃)C₄H₇(CH₃)₂. R⁸ can be hydrogen, hydroxy, methyl, lower alkyl, halo, alkoxy, or acyloxy. R⁹ can be hydrogen, methyl, lower alkyl, halo, perhalomethyl, hydroxy, alkoxy, or acyloxy. R¹⁰ can be hydrogen, hydroxy, methyl, lower alkyl, halo, alkoxy, or acyloxy. Each of rings A, B, C and D, independently, can be saturated, contains at least one double bond, or is aromatic.

In some embodiments, the hydrophobic phase can include a surfactant, a sterol, and a wall forming material. The sterol can be estradiol, testosterone, progesterone, cholesterol, dehydroepiandrosterone, fludrocortisone, or cortisol.

In certain embodiments, the hydrophobic phase includes a surfactant, a membrane modulator, a wall forming material, and an oil. The oil can be soybean oil, avocado oil, squalene oil, sesame oil, olive oil, canola oil, mink oil, cotton seed oil, palm oil, corn oil, rapeseed oil, safflower oil, coconut oil, linseed oil, jojoba bean oil, palm oil, sunflower oil, fish oils, wheat germ oil, almond oil, apricot seed oil, macadamia nut oil or peanut oil.

The details of one or more embodiments are set forth in the description below. Other features, objects, and advantages will be apparent from the description and from the claims.

### BRIEF DESCRIPTION OF THE DRAWINGS

FIGS. 1A and 1B are graphs depicting a linear plot (FIG. 1A) and semi-logarithmic plot (FIG. 1B) of testosterone serum concentration-time profiles over 168 hours in a first rabbit.

FIGS. 2A and 2B are graphs depicting a linear plot (FIG. 2A) and semi-logarithmic plot (FIG. 2B) of testosterone serum concentration-time profiles over 168 hours in a second rabbit.

### DETAILED DESCRIPTION

The composition is an emulsion or other microscopic structure formed with a membrane modulator as part of each individual particle of the emulsion or structure including a hydrophilic phase and a hydrophobic phase. The composition appears homogeneous to the unaided eye.

The composition is formed when a hydrophobic phase is contacted with a hydrophilic phase. The method of contacting can include a high shear emulsification procedure. The amount of shear necessary to form a stable composition can vary depending on the components used.

The product formed during contacting is an opaque fluid, lotion, or cream-like substance that can be a non-Newtonian fluid properties. A non-Newtonian fluid is a fluid whose shearing stress is not proportional to the shearing rate. The proportionality constant is the non-Newtonian viscosity.

A stable composition is a composition that remains visually homogenous, with no aggregation or other phase separation visible to the unaided eye. The composition can be a white or slightly off-white cream with no visible chunks or excess hydrophobic or hydrophilic phase. The composition exhibits long-term visual stability. The composition does not visibly separate into immiscible phases after at least three months, preferably at least six months, and more preferably at least one year. For example, a stable composition appears three months later, as it did when it was first made.

The composition can be an emulsion, for example, an oil-in-water dispersion or another lipid structure such as a small vesicle (for example lipid sphere which often consists of several substantially concentric bilayers separated from each other by layers of aqueous phase), a micelle or a lamellar phase. The composition can form as a result of hydrophobic forces which drive apolar residue (i.e., long hydrocarbon chains) away from water and drive polar head group toward water when a hydrophobic oil phase is mixed with an hydrophilic phase. The other lipid structure includes, but is not limited to, unilamellar, paucilamellar, and multilamellar, micellar or hexagonal phase.

In general, the hydrophobic phase can include a membrane modulator and a wall forming material. The composition can form a membrane-like structure upon contacting the hydrophobic and hydrophilic phases. When hydrophobic phases and hydrophilic phases are contacted, hydrophobic forces drive apolar residues of the membrane modulator and the wall forming materials (i.e. long hydrocarbon chains) away from water, forming the membrane-like structure. The resultant composition can function as a delivery system by delivering the membrane modulator in the membrane to a intracellular site in the target cell.

The composition can include a wall forming material for example, a surface-active agent. The surface-active agent can include a long chain heteroatom-containing compound. The wall forming material and amphiphile can include, for example, a fatty acid alcohol, a polyoxyethylene fatty ester, a polyoxyethylene fatty acid ether, a diethanolamine, a long-chain acyl amide, a long-chain acryl amino acid amide, a long chain acyl amide, a polyoxyethylene sorbitan oleate, a polyoxyethylene glyceryl monostearate or polyoxyethylene glyceryl monoleate, or mixture, analog and derivative thereof. The composition can include a surfactant. The surfactant can be a polyoxyethylene fatty acid ester or a polyoxyethylene fatty acid ether or polyoxyethylene sorbitan fatty acid ester or sorbitan fatty acid ester, or mixture, analog and derivative thereof.

The wall forming material can include a polyoxyalkylene fatty acid ether or a polyoxyalkylene fatty acid ester. A suitable polyoxyalkylene fatty acid ether can have the formula

R¹—O(C₂H₄O)ₘH;

where R¹ is a C₆-C₃₀ alkyl, alkenyl, or alkdienyl group, for example, lauric, myristic, cetyl, stearic or oleic acid, single or double unsaturated octadecyl acids or double unsaturated eicodienoic acid or their derivative, and m is an integer range from 2-30. For example, R¹ is a C₁₂-C₁₈ alkyl, alkenyl, or alkdienyl group and m is an integer range from 2-10. Example of such a wall forming material can include surfactant, for example, a polyoxyethylene(m) stearyl ether, where m is the number of oxyethylene units in the polyethylene oligomer, such as polyoxyethylene(2) stearyl ether (in which the average m is 2), polyoxyethylene(10) stearyl ether (in which m is 10) or polyoxyethylene(2) oleyl ether (in which the average m is 2).

A suitable polyoxyalkylene fatty acid ester can have the formula

R²—COO(C₂H₄O)ₙH

where R² is a C₆-C₃₀ alkyl, alkenyl, or alkdienyl group, for example, lauric, myristic, cetyl, stearic or oleic acid, single or double unsaturated octadecyl acid or double unsaturated eicodienoic acid or their derivative, and n is an integer range from 2-30. For example, R² is a C₁₂-C₁₈ alkyl, alkenyl, or alkdienyl group and n is an integer range from 10-20. Examples of such a wall forming material can include surfactant, for example, a polyoxyethylene(n) stearyl ester, where n is the number of oxyethylene units in the polyethylene oligomer, such as polyoxyethylene (20) sorbitan monostearate (in which the average n is 20) or polyoxyethylene (20) sorbitan monooleate (in which the average n is 20). The surfactant can include sorbitan monostearate, sorbitan monoloeate and sorbitan monolaurate, which can be used for a wall forming material. Examples of suitable surfactants are commercially available from Uniqema (Wilmington, Del., USA).

The wall forming material can include a long or complex chain alcohol, such as a C₁₂-C₁₈ fatty alcohol, for example, cetyl, stearyl, chimyl or batyl alcohol. Glycerol derivative can also be included as a wall forming material, for example, glycerol monostearate, glycerol monooleate, glycerol monomyristate and glycerol monocarylate. Additional example can include palm butter, cocoa butter, or a caprol.

The composition can include an amphiphile which can provide a charge source and also function as a wall forming material. The charge source can be either a positive or negative charge-producing material. The charge-producing material can be added in the hydrophobic phase to impart a charge that can be either negative or positive. A negative charge can be imparted by a long chain acid having a C₈-C₁₈ alkyl, alkenyl or alkdienyl chain. The long chain acid can be saturated or unsaturated and can include cycloalkyl or aromatic ring substitutent. Preferred negative charge producing material can include oleic acid, stearic acid, palmitic acid, cetyl sulfate, retinoic acid, and analog, mixture and derivative thereof. A positive charge is provided by a cationic halogen-containing compound having a C₁₂-C₁₈ alkyl, alkenyl or alkdienyl chain. In order to produce a net positive charge, various materials such as a long chain amine, a quaternary ammonium compound and a long chain pyridinium compound, for example, cetylpyridinium chloride, cetyl trimethyl ammonium bromide, and analog, mixture and derivative thereof, can be included.

The membrane modulator can be, in general, a lipohilic molecule that plays an active function in regulating, controlling or causing a desired physiological outcome in a mammal. A membrane modulator is a material which participates in facilitating membrane formulation. For example, the membrane modulator can be a structural compound found in a biological membrane such a a eukaryotic, prokaryotic or archae cell membrane. The membrane modulator can be a compound with a carbocyclic or heterocyclic ring structure containing 3-20 atoms in the ring. The ring can be aliphatic, unsaturated or aromatic. The membrane modulator can have a multi-ring structure, such as a fused ring structure. The fused ring structure can be a bicyclic, tricyclic, or quadracyclic structure.

The membrane modulator can be a biologically active compound. For example, the membrane moduclator can be a drug, sterol, antibiotic or other therapeutic compound. The membrane modulator can be a sterol from eukaryotic source, such as animal or plant source, a prokaryotic source or archae source. A membrane modulator can be a modified sterol defined as a derivative of a natural sterol. One class of sterols has the formula:

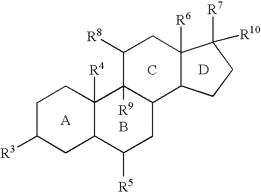

R³ can be hydroxy, acetoxy, propionoxy, methoxy, acyloxy, lower alkyloxy or benzoyloxy, or carbonyl.

Each of R⁴ and R⁵ independently can be, hydrogen, methyl, lower alkyl, halo, alkoxy, or acyloxy.

R⁶ can be methyl, lower alkyl, hydroxy, halo, alkoxy, or acyloxy.

R⁷ can be hydrogen, carbonyl, halo, hydroxy, alkoxy, acyloxy, oxo, —C(O)CH₃, or —C(O)CH₂OH or —CH(CH₃)C₄H₇(CH₃)₂.

R⁸ can be hydrogen, hydroxy, methyl, lower alkyl, halo, alkoxy, or acyloxy.

R⁹ can be hydrogen, methyl, lower alkyl, halo, perhalomethyl, hydroxy, alkoxy, or acyloxy.

R¹⁰ can be hydrogen, hydroxy, methyl, lower alkyl, halo, alkoxy, or acyloxy.

Each of rings A, B, C and D independently is saturated, contains at least one double bond, or is aromatic.

Examples of sterols can include the following or their analog, mixture and derivative thereof:

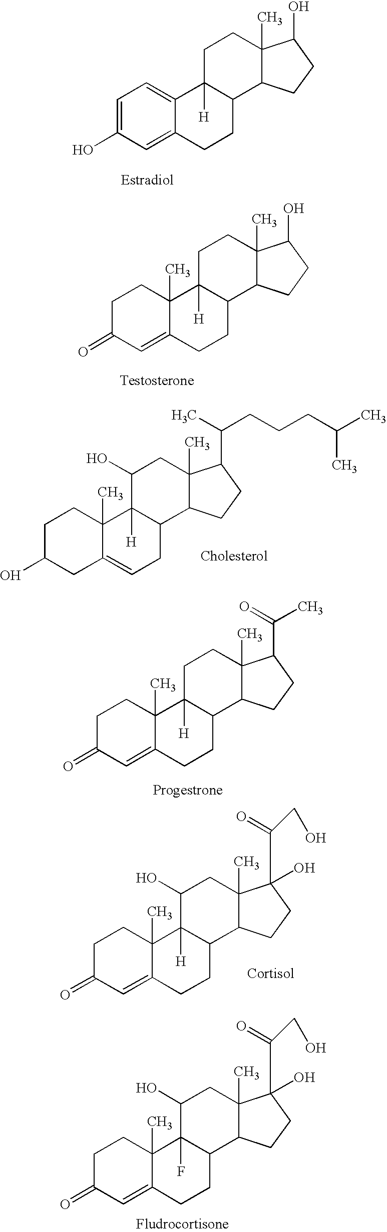

The membrane modulator can be a plant sterol. A plant sterol is known to help in lowering serum cholesterol by inhibiting cholesterol absorption in the digestive system. The plant sterol as a membrane modulator in the composition and can include a plant sterol and a plant stanol (hydrogenated form of plant sterols), for example, beta-sitosterol (24-ethyl-5cholestene-3-beta-ol), its hydrogenated form, beta-sitostanol (24-ethyl-5-alpha-cholestane-3-beta-ol), campesterol, brassicasterol and stigmasterol. The plant sterols are generally commercially available.

The membrane modulator can include an antibiotic. Such antibiotic membrane modulator can be used to inactivate bacteria, particularly gram negative bacteria such as Helicobacter pylori, Escherichia coli, Shigella flexneri or Salmonella enteriditis upon contact. The composition may be used to inactivate or prevent infection secondary to Streptococcus pneumoniae, Group A beta-hemolytic Streptococcus, Haemophilus influenzae, and Neissaria meningitidis. The composition can be administered orally to inactivate or prevent gastrointestinal infection such as gastritis or peptic ulcer disease secondary to Helicobacter pylori. The composition can also be used to inactivate or prevent infection secondary to Neissaria gonorrhoeae, Gardenella vaginalis and Group B Streptococcus. The composition may also be used for dermatological application as a cream or gel to inactivate or prevent infection secondary to Propionibacteriul acnes, Staphylococcus aureus, Staphylococcus epidermidis, and Group B Streptococcus. An illustrative example that is suitable includes an antibacterial agent or an anti-infective agent, for example, ciprofloxacin, cefuroxime, cefatrizine, cefpodoxime, clarithromycin, loracarbef, azithromycin, cefixime, cefadroxil or amoxycillin. An anti-infective agent is an agent which treats or prevents infection at a specified body site due to a specified, susceptible microorganism(s). An antiviral agent, for example, can include acyclovir; an immunosuppressant can, for example, include cyclosporin and tetracyclic triterpene. Broad-spectrum antibiotic can include, for example, cephalosporin; an antiprotozoal can include, for example, 2- metronidazole or 5-metronidazole. Skeletal muscle relaxants can include, for example, baclofen. The drug itself or its pharmaceutically acceptable salt or ester may be used in the present invention.

Other examples of a membrane modulator include creatine, miconazole nitrate, aspirin, and precursor of steroid such as dehydroepiandrosterone (DHEA), a amino acid and a peptide or a drug, or analog, mixture and derivative thereof. Other classes of membrane modulators can include amino acids.

Oils useful in forming the composition include a broad spectrum of water-immiscible materials, such as soyabean oil, avocado oil, squalene oil, sesame oil, olive oil, canola oil, mink oil, cotton seed oil, palm oil, corn oil, rapeseed oil, safflower oil, coconut oil, linseed oil, jojoba bean oil, palm oil, sunflower oil, wheat germ oil, nut oil, for example, almond oil, apricot seed oil, macadamia nut oil or peanut oil, or mixture, analog and derivative thereof.

The hydrophilic phase can be purified water or water for injection, but can include any of a variety of mixtures of water with alcohol up to 70% alcohol solution. The hydrophilic phase can also be any of a number of different saline solutions including, for example, a phosphate buffered saline.

The hydrophobic phase is mixed with a hydrophilic phase, for example, under shear mixing conditions, to form the composition. Mixing can include, for example, high speed mixing or blending. Shear mixing is mixing of the hydrophobic phase with the hydrophilic phase under turbulent or shear conditions which provide adequate mixing of the two phases. Pump speed can be modified depending on the viscosity of the materials and the size of the orifices selected. Shear mixing can be achieved by liquid shear which is substantially equivalent to a relative flow rate for the combined phases of, for example, 5-30 m/s through a 1 mm radius orifice.

The hydrophilic content of the composition can vary depending on components from 92% hydrophilic phase to 8% hydrophilic phase by weight with the remaining content being composed of one of a variety of hydrophobic phase formulations.

In one example, a surfactant, a charged molecule and testosterone were combined with water to form a non-Newtonian fluid that was 60% water by weight, had a final testosterone concentration equal to approximately 100 mg/g (10%), a surfactant concentration of 29.2%, and an oleic acid concentration of 0.8%. The melted hydrophobic phase was combined with the hydrophilic phase using a high shear mixer or other instrument designed to promote solution mixing or homogenization. The high shear mixer can be, for example, one used in a flow or batch type process with a rotor-stator type probe emulsifier.

In another example, stearyl alcohol, polyoxyethylene (20)sorbitan monooleate polyoxyethylene(20) sorbitan monooleate and progesterone were combined with water to form a non-Newtonian fluid that was 80% water by weight, 15% progesterone, and 2.5% stearyl alcohol and polyoxyethylene(20) sorbitan monooleate respectively. The melted hydrophobic phase was combined with the water using a high shear emulsification process.

The composition can include a cream, a gel or a pharmaceutically acceptable carrier, for example, an aqueous syringeable saline solution to allow appropriate administration of the composition.

A possible route for administration of the composition to a mammal can occur through one of the many standard routes of administration which include intravenous, intranasal, intramuscular, intramucosal, subcutaneous, percutaneous, intratracheal, orally and topical administration. For example, one method of administration for a formulation of the composition containing testosterone would be to inject the dose subcutaneously or intramuscularly. The dose could then slowly break down within the body and serve as a method of time release delivery of the testosterone for those that need testosterone replacement treatment. This injection could occur through use of a 25 gauge or thinner bore needle, or through use of a needleless injection system.

Alternatively, the composition could be administered orally and be broken down within the gastrointestinal track, releasing the biologically active compound for use by the body. This form of delivery could also be used to treat gastrointestinal illnesses such as peptic ulcers, or Helicobacter pylori infections. An additional route of delivery can be by intramucosal route. The composition could be inserted into any of the accessible mucosa for either systemic or localized treatment and delivery. For example, a progesterone-containing composition could be applied to the vaginal mucosa to assist women in need of progesterone replacement. Alternatively, an antibiotic-containing composition may be useful in treating mucosal infections, and an anti-asthmatic-containing composition sprayed into the lungs. Additional routes of administration could include intravenous, intramuscular and subcutaneous routes of delivery. Finally, localized delivery, by spreading topically or in conjunction with an applicator or medical cloth or bandage containing or spread with the composition could be used to treat external medical problems. An example, would be a hydrocortisone containing composition for application on poison oak, poison ivy, and insect and spider bites. Alternatively, the composition may be administered percutaneously by catheterization, for example, a catheter can be inserted into an artery or vein or catheter can be inserted into a particular organ to deliver the composition to that location.

A number of embodiments of the invention have been described. Nevertheless, it will be understood that various modifications may be made without departing from the spirit and scope of the invention.

### EXAMPLE 1

This example illustrates the formation of a testosterone composition. In this example, stearyl alcohol, polyoxyethylene(20) sorbitan monooleate, testosterone and water are co-dissolved to form the hydrophobic phase. The stearyl alcohol and the polyoxyethylene(20) sorbitan monooleate were heated together until completely melted. Next, the testosterone was added to the melted components and heated until melted. The ratio of the components by weight in the hydrophobic phase was 1:1:5 mixture of polyoxyethylene(20) sorbitan monooleate:testosterone. The hydrophilic phase in this example was water. The resulting solution was emulsified with a volume of water using a high shear emulsifier with fine screen, until visibly homogenous. A non-Newtonian fluid product was formed with a final weight percent of water equal to 80%, a final weight percent of lipid or hydrophobic phase of 20%, and a final concentration of active ingredient (testosterone) of approximately 143 mg/g final product. Other examples of with similar composition have been prepared with a ratio of components in the hydrophobic phase of 1:1:6 stearyl alcohol: polyoxyethylene(20) sorbitan monooleate, resulting in a final active concentration of 150 mg/g final product of testosterone.

### EXAMPLE 2

This example illustrates the formation of an estrogen composition. In this example, polyoxyethylene(2) stearyl ether, oleic acid, estrogen (17-β estradiol) form the hydrophobic phase, as describe in Example 1. The polyoxyethylene(2) stearyl ether and oleic acid were weighed and completely melted together. Next, the estrogen was added to the melted components and heated until completely melted. The ratio of the components by weight in the hydrophobic phase was approximately 14.9 : 0.1:5.0 of polyoxyethylene(2) stearyl ether, oleic acid and estrogen (1 7-β estradiol). The hydrophilic phase was 40% ethanol in water (actually 40 %×95% since the non-denatured alcohol source is 190 proof). The hydrophobic phase was mixed with the hydrophilic phase using a high shear emulsifier until visibly homogeneous. The final non-Newtonian fluid product was 60% hydrophilic phase and the final concentration of active ingredient (estrogen) was approximately 100 mg/g.

### EXAMPLE 3

In this example, a progesterone containing composition was prepared. In this example, polyoxyethylene(10) stearyl ether, oleic acid, progesterone and vitamin E form the hydrophobic phase. The ratio of the components by weight in the hydrophobic phase was approximately 39.48: 1.75: 14.1: lofpolyoxyethylene 10 stearyl ether, oleic acid, progesterone and vitamin E. The polyoxyethylene(10) stearyl ether and oleic acid were weighed and completely melted. Next the progesterone was added and heated until completely melted. Finally, the Vitamin E was added. The hydrophilic phase was water. The hydrophobic phase was mixed with the hydrophilic phase using a high shear emulsifier until visibly homogeneous. The final non-Newtonian fluid product was 60% water and the final concentration of the progesterone was approximately 100 mg/g.

### EXAMPLE 4

This example illustrates the formation of a progesterone composition. In this example, glycerol monostearate (GMS), progesterone, polyoxyethylene(20) sorbitan monostearate, cetylpyridinium chloride (CPC) form the hydrophobic phase. The ratio of components by weight in the hydrophobic phase was approximately 4.1:2.22:1:1.57 glycerol monostearate (GMS), progesterone, polyoxyethylene(20) sorbitan monostearate and cetylpyridinium chloride (CPC). The hydrophilic phase was water. The GMS, polyoxyethylene(20) sorbitan monostearate, and CPC were weighed and melted completely. Next, the progesterone was added and heated until it was melted completely. The hydrophobic phase was then mixed with the hydrophilic phase using a high shear emulsifier until visibly homogeneous. The final non-Newtonian fluid product was 60% water and the final concentration of the progesterone was approximately 100 mg/g.

### EXAMPLE 5

In this example, a testosterone containing composition was prepared. In this example, stearyl alcohol, polyoxyethylene(20) sorbitan monooleate, testosterone, cholesterol and water. The hydrophobic phase was composed of stearyl alcohol, polyoxyethylene(20) sorbitan monooleate, testosterone and cholesterol. The ratio of the components by weight in the hydrophobic phase was 1:1:3:2 stearyl alcohol, polyoxyethylene(20) sorbitan monooleate, testosterone and cholesterol respectively. The hydrophilic phase in this example was water. polyoxyethylene(20) sorbitan monooleate, and cholesterol were heated together until completely melted. Next, the testosterone was added to the melted components and heated until melted. This hydrophobic phase was emulsified with the hydrophilic phase, using a high shear emulsifier until visibly homogenous to form a non-Newtonian product with a final weight percent of water equal to 80%, a final weight percent of lipid or hydrophobic phase of 20%, and a final concentration of active ingredient (testosterone) of approximately 85 mg/g final product.

### EXAMPLE 6

This example illustrates a progesterone containing composition. In this example, polyoxyethylene(2) stearyl ether, oleic acid, cholesterol, and progesterone form the hydrophobic phase. The ratio of the components by weight in the hydrophobic phase was approximately 14.9:0.1:2.5:2.5 of polyoxyethylene(2) stearyl ether, oleic acid, cholesterol, and progesterone. The hydrophilic phase was water. The polyoxyethylene(2) stearyl ether, oleic acid, and cholesterol were weighed and completely melted. Next, the progesterone was added to the melted components and heated until completely melted. The hydrophobic phase was mixed with the hydrophilic phase using a high shear emulsifier until visibly homogeneous. The final non-Newtonian fluid product was 60% hydrophilic phase and the final concentration of active ingredient (progesterone) was approximately 50 mg/g.

### EXAMPLE 7

In this example, a testosterone formulation was prepared. The glycerol monostearate (GMS), polyoxyethylene(20) sorbitan monostearate, testosterone and DHEA. The ratio of the components by weight was approximately 4.15:1:2.58:2.58 of glycerol monostearate (GMS), polyoxyethylene(20) sorbitan monostearate, testosterone and DHEA. The GMS and polyoxyethylene(20) sorbitan monostearate were weighed ad completely melted, the testosterone and DHEA were added and the hydrophobic phase was heated until completely melted. The hydrophilic phase was phosphate buffered saline solution. The hydrophobic phase was mixed with the hydrophilic phase using a high shear emulsifier until visibly homogeneous. The final non-Newtonian fluid product was 60% hydrophilic phase and the final concentration of active ingredients (testosterone and DHEA) was approximately 100 mg/g each.

### EXAMPLE 8

This example illustrates a formulation containing testosterone with near extreme maximum water content. In the example, batyl or chimyl alcohol, testosterone and polyoxyethylene(20) sorbitan monooleate form the hydrophobic phase. The ratio of the components by weight was 1:2:1 of batyl or chimyl alcohol, testosterone, and polyoxyethylene(20) sorbitan monooleate. The alcohol and surfactant were heated until melted; the testosterone was added subsequently and melted completely. The hydrophilic phase was water. The hydrophobic phase was mixed with the hydrophilic phase using a high shear emulsifier until visibly homogenous. The non-Newtonian fluid product was 92% water and the final concentration of active ingredient (testosterone) was 40 mg/g.

### EXAMPLE 9

This example shows a progesterone containing formulation with an extreme minimum of water content. In this example, polyoxyethylene(2) oleyl ether, oleic acid, and progesterone form the hydrophobic phase. The ratio of the components in the lipid phase was 9.82:1:8.94 of polyoxyethylene(2) oleyl ether, oleic acid, and progesterone. The polyoxyethylene(2) oleyl ether and the oleic acid were already liquid, so they were heated to just below the melting point of the progesterone at which point the progesterone was added and melted completely. The hydrophobic phase was mixed with the hydrophilic phase using a high shear emulsifier until visibly homogenous. The non-Newtonian fluid product was 20% water and the final concentration of active ingredient (progesterone) was approximately 360 mg/g.

### EXAMPLE 10

This example shows a metronidazole containing formulation. In this example, stearyl alcohol, metronidazole, and polyoxyethylene(20) sorbitan monooleate form the hydrophobic phase and the hydrophilic phase is purified water. The ratio of the components in the lipid phase was 1:5:1 of stearyl alcohol: metronidazole: polyoxyethylene(20) sorbitan monooleate. The stearyl alcohol and the polyoxyethylene(20) sorbitan monooleate were combined and melted. The metronidazole was subsequently added and melted completely. The hydrophobic phase was mixed with the hydrophilic phase using a high shear emulsifier until visibly homogenous. The non-Newtonian fluid product was 80% water and the final concentration of active ingredient (metronidazole) was approximately 142 mg/g.

### EXAMPLE 11

This example illustrates a formulation containing aspirin as the active ingredient. In this example, stearyl alcohol, aspirin and polyoxyethylene(20) sorbitan monooleate form the hydrophobic phase. The ratio of the components in this phase was 1:5:1 of stearyl alcohol, aspirin and polyoxyethylene(20) sorbitan monooleate. The stearyl alcohol and the polyoxyethylene(20) sorbitan monooleate were combined and melted. The aspirin was subsequently added and melted completely. The hydrophobic phase was mixed with the hydrophilic phase using a high shear emulsifier until visibly homogenous. The non-Newtonian fluid product was 80% water and the final concentration of active ingredient (aspirin) was approximately 142 mg/g.

### EXAMPLE 12

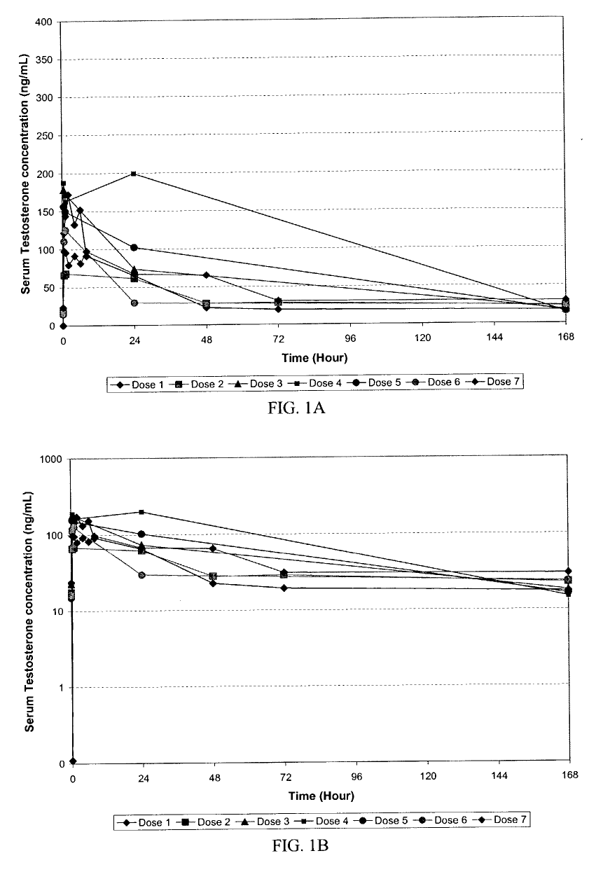

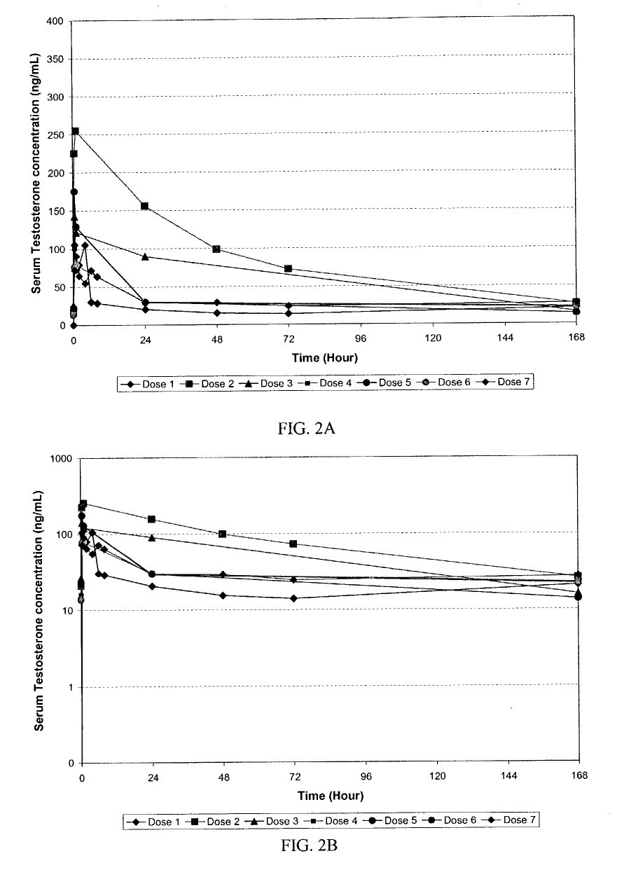

Six, male, castrated (well-healed) rabbits received once weekly, 75 mg, subcutaneous injections of the testosterone formulation of Example 1 (75 mg/0.5 mL) for seven doses. The days of dosing were designated 0, 7, 14, 21, 28, 35 and 42. Animals were approximately seven weeks of age when the first dose was administered. Blood samples for serum harvesting were collected at 4 days prior to dosing, and at 0 (baseline), 0.5, 1, 2, 4, 6, 8, 24, 48, 72, and 168 hours after the first dose. Following the second to sixth doses, serum samples were obtained at 0 (baseline), 0.5, 1, 24, 48, 72 and 168 hours. Following the seventh dose, serum samples were obtained at 0 (baseline), 0.5, 1, 2, 4, 6, 8, 24, 48, 72, 168, 192, 216, 240, 336, 408, 504, 576 and 672 hours post-dose. Serum testosterone concentrations were determined using a commercial kit, “Coat-A-Count® Total Testosterone,” DPC® (Diagnostic Products Corporation, Los Angeles, Calif.), which is a solid-phase radioimmunoassay, based on testosterone-specific antibody immobilized to the wall of a polypropylene tube. FIGS. 1A, 1B, 2A and 2B illustrate both linear plots (FIGS. 1A and FIG. 2A) and semi-logarithmic plots (FIG. 1B and FIG. 2B) of testosterone serum concentration-time profiles over 168 hours (7 days, the interval between doses), in each of two rabbits (FIGS. 1A and 1B for one rabbit and FIGS. 2A and 2B for the second rabbit), for each of the seven doses.

Pharmacokinetic parameters were determined by non-compartmental methods (see, Gibaldi M and Perrier D, “Pharmacokinetics,” 2^{nd} ed. New York: Marcel Dekker, (1982) which is incorporated by reference in its entirety) using the SAS program (“SAS Language Reference: Concepts,” SAS Institute, Cary, N.C., 1999). The following parameters were determined: (1) the maximum serum concentration, Cmax; (2) the time at which Cmax was attained, Tmax; (3) the minimum serum concentration, Cmin; (4) the area under the serum concentration-time curve from time 0 to the last measurable time-point (t), AUC(0-t) (calculated using the linear trapezoidal rule); (5) the area under the serum concentration-time curve from time 0 to ∞, AUC(0-∞), which was calculated by extrapolating the area from the last applicable data point to ∞ by dividing its concentration by the terminal exponential rate constant and adding the extrapolated area to AUC(0-t) to provide AUC(0-∞); (6) the area under the serum concentration-time curve over a dosing interval of 7 days, using the linear trapezoidal rule, denoted AUC(0-168); (7) the average concentration over a dosing interval, Cav, which is equal to (AUC(O-τ)/τ); (8) the dosage form index, DFI, which is equal to ((Cmax-Cmin)/Cav); (9) the terminal exponential half-life, T-½, which is calculated from ln(2)/λ, where λ is the terminal exponential rate constant determined by linear regression of logarithmically transformed terminal exponential data (calculated at 48, 72 and 168 hours); (10) the extravascular (sub-cutaneous) clearance, CL/F, where CL is clearance (L/day/kg) and F is the absolute bioavailability, CL/F being equal either to Dose/AUC(0-∞) for a single-dose, or Dose/AUC(0-t) at steady-state (inasmuch as testosterone concentrations at the beginning of the experiment were close to zero, the contribution of endogenous testosterone production was ignored in this calculation); and (11) absolute bioavailability, F, calculated as CL/(CL/F), where male, rabbit testosterone clearance was taken as 42 liters/day/kg (see, Bourget et al. “Steroid dynamics in the rabbit,” Steroids 43:225-233 (1984), which is incorporated by reference in its entirety). Tables I and II collate the pharmacokinetic parameters following administration of the first dose and the seventh dose, respectively.

TABLE I

| | AUC (0-t) | AUC (0-∞) | Cmax | Tmax | T-1/2 | CL/F | % F (Absolute |
|---|---|---|---|---|---|---|---|
| EXAMPLE | (ng-h/mL) | (ng-h/mL) | (ng/mL) | (hours) | (hours) | (L/day/kg) | Bioavailability) |
| A | 5630.98 | 14982.33 | 171.80 | 2.00 | 374.70 | 34.39 | 122 |
| B | 3352.80 | ND | 105.20 | 0.50 | ND | ND | ND |
| C | 4967.66 | * | 145.60 | 2.00 | * | * | * |
| D | 6308.35 | 6838.61 | 174.00 | 6.00 | 17.30 | 76.08 | 55.2 |
| E | 10388.00 | 10772.53 | 372.40 | 2.00 | 37.50 | 46.35 | 90.6 |
| F | 3661.98 | ND | 102.40 | 2.00 | ND | ND | ND |
| Mean | 5718.29 | 10864.49 | 178.57 | 2.42 | 143.17 | 52.28 | 89.31 |
| SD | 2551.45 | 4072.64 | 99.91 | 1.86 | 200.77 | 21.46 | 33.47 |
| % CV | 44.6 | 37.49 | 55.90 | 76.80 | 140.23 | 41.06 | 37.48 |
| Median | 5299.32 | 10772.53 | 158.70 | 2.00 | 37.50 | 46.35 | 90.61 |
| Min | 3352.80 | 6838.61 | 102.40 | 0.50 | 17.30 | 34.39 | 55.21 |
| Max | 10388.00 | 14982.33 | 372.40 | 6.00 | 374.70 | 76.08 | 122.11 |

* These values were artifacts, due to the very poor estimation of T-1/2 (2100 hours), which in turn affected AUC (0-∞), CL/F and F.
ND = not determinable.

TABLE II

| | AUC (0-168) | Cav | Cmax | Cmin | | Tmax | T-1/2 | CL/F | % F (Absolute |
|---|---|---|---|---|---|---|---|---|---|
| EXAMPLE | (ng-h/mL) | (ng-h/mL) | (ng/mL) | (hours) | DFI | (hours) | (hours) | (L/day/kg) | Bioavailability) |
| A | 7645.28 | 45.51 | 97.40 | 23.44 | 1.63 | 0.50 | 147.5 | 61.7 | 68.1 |
| B | 5081.76 | 30.25 | 90.20 | 22.50 | 2.24 | 1.00 | ND | 95.1 | 44.2 |
| C | 6968.41 | 41.48 | 116.80 | 18.95 | 2.36 | 0.50 | 83.5 | 73.5 | 57.1 |
| D | 10883.07 | 64.78 | 186.40 | 22.40 | 2.53 | 2.00 | 68.0 | 46.7 | 90.0 |
| E | 9506.87 | 56.59 | 145.80 | 25.97 | 2.12 | 1.00 | 86.6 | 55.6 | 75.5 |
| F | 15035.05 | 89.49 | 195.00 | 29.18 | 1.85 | 8.00 | 55.0 | 33.4 | 125.8 |
| Mean | 9186.74 | 54.68 | 138.60 | 23.74 | 2.12 | 2.17 | 88.1 | 61.0 | 76.8 |
| SD | 3502.31 | 20.85 | 44.79 | 3.49 | 0.33 | 2.91 | 35.5 | 21.5 | 28.6 |
| % CV | 38.10 | 38.10 | 32.30 | 14.70 | 15.70 | 134.30 | 40.3 | 35.3 | 37.3 |
| Median | 8576.08 | 51.05 | 131.30 | 22.97 | 2.18 | 1.00 | 83.5 | 58.7 | 71.8 |
| Min | 5081.76 | 30.25 | 90.20 | 18.95 | 1.63 | 0.50 | 55.0 | 33.4 | 44.2 |
| Max | 15035.05 | 89.49 | 195.00 | 29.18 | 2.53 | 8.00 | 147.5 | 95.1 | 125.8 |

ND = not determinable.

Terminal exponential half-lives determined from data at 48, 72 and 168 hours following Doses 1 and 7 were 40 - 80 hours. The true testosterone, disposition half-life in rabbits is in the vicinity of 15 minutes. Even though the system is nonlinear, absolute bioavailability values were calculated by a method assuming linearity. The amount of testosterone systemically available from the dosage form suggest good bioavailability. The relatively low values of the dosage form index (DFI) at all doses suggests this is an excellent sustained-release (SR) product. The data are consistent with a “flip-flop” model (see, Boxenbaum, H., “Pharmacokinetic Tricks and Traps: Flip-Flop Models,” J. Pharm. Pharmaceut. Sci. 1:90-91 (1998), which is incorporated by reference in its entirety), in which serum concentrations are proportional to rate of absorption.

Other embodiments are within the scope of the following claims.

What is claimed is:

1. A composition comprising:
an emulsion comprising a hydrophobic phase and a hydrophilic phase, wherein the hydrophobic phase comprises:
a membrane modulator; and
a wall forming material.

2. The composition of claim 1 wherein the range of ratios of hydrophobic phase to hydrophilic phase comprises from about 1:1.5 to about 11.5:1.

3. The composition of claim 1 wherein the membrane modulator is a sterol.

4. The composition of claim 1 wherein the membrane modulator is a compound of the formula: 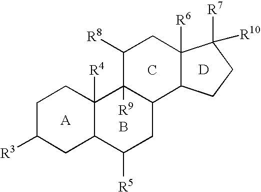
wherein R³ is hydroxy, acetoxy, propionoxy, methoxy, acyloxy, lower alkyloxy, benzoyloxy, or carbonyl;
each of R⁴ and R⁵, independently, is hydrogen, methyl, lower alkyl, halo, alkoxy, or acyloxy;
R⁶ is methyl, lower alkyl, hydroxy, halo, alkoxy, or acyloxy;
R⁷ is hydrogen, carbonyl, halo, hydroxy, alkoxy, acyloxy, oxo, —C(O)CH₃, —C(O)CH₂OH or —CH(CH₃)C₄H₇(CH₃)₂;
R⁸ is hydrogen, hydroxy, methyl, lower alkyl, halo, alkoxy, or acyloxy;
R⁹ is hydrogen, methyl, lower alkyl, halo, perhalomethyl, hydroxy, alkoxy, or acyloxy; and
R¹⁰ is hydrogen, hydroxy, methyl, lower alkyl, halo, alkoxy, or acyloxy,
wherein each of rings A, B, C and D, independently, is saturated, contains at least one double bond, or is aromatic.

5. A composition comprising:
a non-Newtonian fluid comprising a hydrophobic phase in contact with a hydrophilic phase, wherein the hydrophobic phase comprises:
a biologically active compound; and
a wall forming material.

6. The composition of claim 5 wherein the biologically active compound is a sterol.

7. The composition of claim 5 wherein the biologically active compound is an animal sterol.

8. The composition of claim 5 wherein the biologically active compound is a plant sterol.

9. A composition comprising:
a non-Newtonian fluid comprising a hydrophobic phase in contact with a hydrophilic phase, wherein the hydrophobic phase comprises:
an amphiphile;
a biologically active compound; and
a wall forming material.

10. The composition of claim 9 wherein the amphiphile has a positive charge.

11. The composition of claim 9 wherein the amphiphile has a negative charge.

12. The composition of 9 wherein the amphiphile is a surfactant.

13. A composition comprising:
a non-Newtonian fluid comprising a hydrophobic phase in contact with a hydrophilic phase, wherein the hydrophobic phase comprises:
a surfactant;
a sterol; and
a wall forming material.

14. The composition of claim 13 wherein the sterol is estradiol, testosterone, progesterone, cholesterol, dehydroepiandrosterone, fludrocortisone, or cortisol.

15. A composition comprising:
a non-Newtonian fluid comprising a hydrophobic phase in contact with a hydrophilic phase, wherein the hydrophobic phase comprises:
a surfactant;
a membrane modulator;
a wall forming material; and
an oil.

16. The composition of claim 15 wherein the oil is soybean oil, avocado oil, squalene oil, sesame oil, olive oil, canola oil, mink oil, cotton seed oil, palm oil, corn oil, rapeseed oil, safflower oil, coconut oil, linseed oil, jojoba bean oil, palm oil, sunflower oil, fish oils, wheat germ oil, almond oil, apricot seed oil, macadamia nut oil or peanut oil.

17. A composition comprising:
a non-Newtonian fluid comprising a hydrophobic phase in contact with a hydrophilic phase, wherein the hydrophobic phase comprises:
a surfactant;
a membrane modulator; and
a charge source.

18. The composition of claim 17 wherein the membrane modulator is a sterol.

19. The composition of claim 17 wherein the membrane modulator is an animal sterol.

20. The composition of claim 17 wherein the membrane modulator is a plant sterol.

21. The composition of claim 17 wherein the charge source has a positive charge.

22. The composition of claim 17 wherein the charge source has a negative charge.

23. The composition of claim 17 wherein the membrane modulator is an antibiotic.

24. The composition of claim 17 wherein the membrane modulator is an amino acid.

25. The composition of claim 17 wherein the membrane modulator is a peptide.

26. The composition of claim 17 wherein the surfactant is polyoxyethylene sorbitan ester, polyoxyethylene fatty acid ether or sorbitan monoester.

27. The composition of claim 1 wherein the membrane modulator is an animal sterol.

28. The composition of claim 1 wherein the membrane modulator is testosterone, progesterone, estrogen, cholesterol, dehydroepiandrosterone, fludrocortisone, or cortisol.

29. The composition of claim 1 wherein the membrane modulator is creatine, miconazole nitrate, aspirin, 2-metronidazole, or 5-metronidazole.

30. The composition of claim 1 wherein the membrane modulator is an antibiotic.

31. The composition of claim 1 wherein the membrane modulator is ciprofloxacin, cefuroxime, cefatrizine, celpodoxime, clarithromycin, loracarbef, azithromycin, cefixime, cefadroxil, amoxycillin, acyclovir, cyclosporin, cephalosporin or baclofen.

32. The composition of claim 1 wherein the membrane modulator is a plant sterol.

33. The composition of claim 32 wherein the plant sterol is a sterol.

34. The composition of claim 32 wherein the plant sterol is a stanol.

35. The composition of claim 32 wherein the membrane modulator is beta-sitostanol (24-ethyl-5 -cholestene-3 -beta-ol), beta-sitostanol (24-ethyl-5 -alpha-cholestane-3 -beta-ol), campesterol, brassicasterol or stigmasterol.

36. The composition of claim 1 wherein the membrane modulator is an amino acid.

37. The composition of claim 1 wherein the membrane modulator is a peptide.

38. The composition of claim 1 wherein the wall forming material is polyoxyethylene sorbitan ester, polyoxyethylene fatty acid ether or sorbitan monoester.

39. The composition of claim 1 wherein the hydrophobic phase comprises polyoxyethylene(20) sorbitan monooleate, stearyl alcohol, and testosterone.

40. The composition of claim 1 wherein the hydrophilic phase comprises an alcohol and water mixture.

41. The composition of claim 1 wherein the hydrophilic phase comprises water.

42. The composition of claim 1 wherein the hydrophilic phase comprises phosphate buffered saline.

43. The composition of claim 1 wherein the membrane modulator comprises a modified sterol.

44. The composition of claim 1 wherein the membrane modulator contains a ring structure.

45. The composition of claim 1 wherein the membrane modulator contains a multi-ring structure.

46. The composition of 1 wherein the membrane modulator is a drug.

47. A method of making a composition comprising liquefying a hydrophobic phase and mixing the hydrophobic phase with a hydrophilic phase to form a non-Newtonian fluid including a membrane modulator.

48. The method of 47 wherein the liquefying comprises melting.

49. The method of 47 wherein the mixing is accomplished using a high shear mixer.

50. The method of 47 wherein the hydrophobic phase is liquefied to form a slurry.

51. The method of 47 wherein the hydrophilic phase is added to the hydrophobic phase while mixing.

52. The method of 47 wherein the hydrophobic phase is added to the hydrophilic phase while mixing.

53. The method of 47 wherein the hydrophobic phase comprises:
a membrane modulator; and
a wall forming material.

54. The method of 53 wherein the membrane modulator is a animal sterol, plant sterol, antibiotic, amino acid, drug or peptide.

55. The composition of claim 54 wherein the membrane modulator is testosterone, progesterone, estrogen, cholesterol, dehydroepiandrosterone, fludrocortisone, or cortisol.

56. The composition of claim 53 wherein the wall forming material is polyoxyethylene sorbitan ester, polyoxyethylene fatty acid ether or sorbitan monoester.

57. A method of delivering a biologically active molecule comprising:
providing a composition comprising a hydrophobic phase and a hydrophilic phase, wherein the hydrophobic phase comprises a membrane modulator and a wall forming material; and
contacting the composition with a human body.

58. The method of 57 wherein contacting includes transdermal delivery.

59. The method of 57 wherein contacting includes intramuscular delivery.

60. The method of 57 wherein contacting includes subcutaneous delivery.

61. The method of 57 wherein contacting includes mucosal delivery.

62. The method of 57 wherein contacting includes oral delivery.

63. The method of 57 wherein contacting includes intravenous delivery.

64. The method of 57 wherein contacting includes time release delivery.

65. The method of 57 wherein contacting includes delivery through a needle having a 25 gauge or thinner bore needle.
